(19) United States
(12) Patent Application Publication
(10) Pub. No.: US 2025/0085174 A1
(43) Pub. Date: Mar. 13, 2025

(54) HIGHLY SENSITIVE HYBRID RESPONSE CAPACITIVE PRESSURE SENSOR

(71) Applicant: BOARD OF REGENTS, THE UNIVERSITY OF TEXAS SYSTEM, Austin, TX (US)

(72) Inventor: Nanshu LU, Austin, TX (US)

(21) Appl. No.: 18/292,725

(22) PCT Filed: Jul. 28, 2022
(86) PCT No.: PCT/US2022/074270

§ 371 Date: Jan. 26, 2024

Related U.S. Application Data
(60) Provisional application No. 63/226,243, filed on Jul. 28, 2021.

(51) Int. Cl. G01L 1/14; A61B 5/024; H10N 30/092; H10N 30/30; H10N 30/85; H10N 30/87

(52) U.S. Cl. CPC G01L 1/142; A61B 5/02444; H10N 30/092; H10N 30/302; H10N 30/852; H10N 30/877; A61B 2562/0247; A61B 2562/12

(57) ABSTRACT

Described herein is a flexible hybrid response pressure sensor (HRPS) composed of an electrically conductive porous nanocomposite (PNC) laminated with an ultrathin dielectric layer, resulting in significantly enhanced sensitivities (i.e., more than 400%) over wide pressure ranges, from 3.13 kPa-1 within 0-1 kPa to 0.43 kPa-1 within 30-50 kPa. In some aspects, the PNC is composed of carbon nanotubes (CNT) and a low viscosity and flexible, strong, and elastic rubber (e.g., Ecoflex™ (Smooth-On, Inc., Macungie, PA-Ecoflex™ is a platinum-catalyzed silicone)), and the ligaments of the PNC are electrically conductive due to adequate CNT doping.

### CROSS REFERENCE TO RELATED APPLICATION

This application claims priority to and benefit of U.S. provisional patent application Ser. No. 63/226,243 filed Jul. 28, 2021, which is fully incorporated by reference herein.

### GOVERNMENT SUPPORT CLAUSE

This invention was made with government support under Grant No. N00014-20-1-2112 awarded by the Office of Naval Research and Grant No. W911NF-19-2-0333 awarded by the Army Research Office. The government has certain rights in the invention.

### TECHNICAL FIELD

Aspects of the disclosure relate generally to an improvement in technology for highly sensitive capacitive pressure sensors over a wide pressure range enabled by the hybrid responses of a highly porous nanocomposite material.

### BACKGROUND

Past research aimed at increasing the sensitivity of capacitive pressure sensors has mostly focused on developing dielectric layers with surface/porous structures or higher dielectric constants. However, such strategies have only been effective in improving sensitivities at low pressure ranges (e.g. up to 3 kPa).

Flexible pressure sensors able to conform to curvilinear and even deformable surfaces are of increasing demand in emerging fields such as robotics, prosthetics, surgical tools, biometric sensors, and more.^{[1-3]} For instance, advanced flexible pressure sensors have been used for robot fingers handling delicate items,^{[4]} artificial gloves distinguishing hand gestures,^{[5-7]} tactile sensing,^{[8, 9]} noninvasive measurement of blood pressure,^{[10]} and artificial vessels capable of detecting pulse waves.^{[11, 12]} Different applications correspond to different pressure ranges: subtle pressures below 1 kPa for ultra-sensitive e-skin capturing soft touch^{[13]} or palpating cardiovascular activity^{[14, 15]}; low pressures between 1-10 kPa for intra-body pressures^{[16, 17]} and pressures associated with daily activities (e.g. gentle manipulation of items)^{[18]}; and high pressures of more than 10 kPa for blood pressure monitoring devices^{[19]} and the plantar pressure of body weight^{[20]}. Sometimes, subtle pressures are even superimposed on high-pressure preloads, such as when pressure sensors are attached to target surfaces using a covering tape or incorporating pressure sensors under other wearable devices. For those applications, flexible pressure sensors with high sensitivity throughout large pressure ranges are desired.

A variety of pressure sensing mechanisms, including piezoresistive,^{[5, 11, 21-23]} piezoelectric,^{[24-26]} capacitive,^{[6, 8, 27-31]} optical,^{[32-33]} and ionic responses ^{[34-35]} have been explored. Under compression, the pressure-sensing materials generate changes in electrical resistance, voltage, capacitance, transmittance of light, or capacitance of an electric double layer, respectively. Piezoresistive pressure sensors have advantages of facile fabrication, simple structure and readout circuits, but suffer from constant power consumption, large confounding temperature sensitivity, and hysteresis.^{[36-38]} In contrast, piezoelectric sensors do not require input power but are only suitable for measuring dynamic pressures such as pulses or vibrations.^{[36-38]} Optical pressure sensors exhibit negligible signal drift but have limitations including complex setup, high power consumption, and signal alteration or attenuation due to bending or misalignment.^{[37, 39]} Ionic pressure sensors can be thin and transparent and possess enormous sensitivity due to the large capacitance change of the electrical double layer.^{[40, 41]} However, ionic sensors are less stable and/or biocompatible,^{[34]} and require a threshold pressure for the ionogel and the electrode to make initial contact.^{[42-44]}

The sensitivity of capacitive pressure sensors mainly depends on the deformation of the dielectric material and is damped as the effective compressive modulus of the dielectric material increases with compression due to fixed boundaries.^{[45]} In pursuit of higher sensitivity, recent research has focused on engineering the dielectric materials by adding air gaps and/or increasing their dielectric constants. Air gaps on the surface or inside of a dielectric material reduce the effective compressive modulus. Moreover, they enable the effective dielectric constant to increase with compression, as the volume fraction of air is replaced by solids with higher dielectric constants.^{[8]} To incorporate air gaps in dielectric materials, strategies including micropatterned surfaces,^{[6, 12, 28, 46-50]} foams,^{[51-56]} nanowire networks,^{[57-58]} fabrics,^{[30, 59]} and spacing layers ^{[27, 31, 60-62]} have been employed. To enhance the effects of air gaps, methods such as coating and doping elastomers with high dielectric constant materials or conductive nanomaterials to achieve high dielectric constant composites have been explored.^{[54, 56, 58, 63-67]} However, these techniques for sensitivity enhancement were only effective over a small pressure range. The effects weaken as the air gaps diminish with compression. After extensive research in the past decade, the improvements of porosity and the dielectric constants have almost reached their limits. A fundamentally new strategy is needed to achieve capacitive pressure sensors with high sensitivity over wide ranges of pressure.

Therefore, what is desired are capacitive pressure sensors with good repeatability, temperature independence, low power consumption, high spatial resolution, and suitability for large-area applications. In particular, capacitive pressure sensors are desired that are highly sensitive over a wide range as enabled by hybrid responses of a highly porous nanocomposite.

### SUMMARY

Disclosed and described herein is a capacitive pressure sensor employing the hybrid piezoresistive and piezocapacitive responses of a highly porous nanocomposite (PNC) to attain high sensitivity over a large pressure range (i.e. 3.13 kPa⁻¹ within 0-1 kPa, 1.65 kPa⁻¹ within 1-5 kPa, 1.16 kPa⁻¹ within 5-10 kPa, 0.68 kPa⁻¹ within 10-30 kPa, and 0.43 kPa⁻¹ within 30-50 kPa). In some aspects, the PNC is composed of carbon nanotubes (CNT) and a low viscosity and flexible, strong, and elastic rubber (e.g., Ecoflex™ (Smooth-On, Inc., Macungie, PA—Ecoflexm is a platinum-catalyzed silicone)), and the ligaments of the PNC are electrically conductive due to adequate CNT doping. The PNC is approximately 86% porous with an open cell structure that enables distributed parasitic capacitance. By adding an ultrathin solid insulating layer between the PNC and one side of the electrode, the whole device becomes capacitive. The overall capacitance of the sensor varies with the impedance of the PNC, which is affected by both the resistance and the capacitance changes of the PNC under compression, therefore it is called a hybrid response pressure sensor (HRPS). The disclosed RPS is flexible and can be inexpensively fabricated without any vacuum facilities or microelectromechanical systems (MEMS) fabrication facilities such as cleanrooms. Although the sensitivity of the disclosed RPS may in some instances decay with compression, the decay is quite mild compared with conventional capacitive pressure sensors; hence, high sensitivity can still be attained at large pressures. Also presented herein is an analysis based on simplified circuit models to fully determine the effects of each of the piezo-responses and moreover, to help determine the optimal CNT doping concentration. It is also demonstrated that the disclosed RPS is able to measure pressures from as small as 0.07 Pa due to drosophila weight to as large as 125 kPa due to footsteps.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1A illustrates a method of fabrication of an exemplary PNC used in a hybrid response pressure sensor (HRPS);

FIG. 1B illustrates a SEM image of an open cell nickel foam template for the fabrication of the PNC;

FIG. 1C illustrates SEM top-view of the PNC, where a tubular ligament of PNC is included as an inset;

FIG. 1D illustrates an exploded schematic illustration of an exemplary HRPS

FIG. 1E is a SEM image of a cross-sectional view of an exemplary HRPS;

FIG. 1F illustrates optical images of an exemplary HRPS (1×1 cm2) bent by a tweezer and on a cylindrical rod;

FIG. 2A illustrates an exemplary pressure-strain curve of an exemplary RPS;

FIG. 2B illustrates resistance-strain curves of exemplary PNCs with different doping ratios of CNT;

FIG. 2C illustrates pressure response of the absolute capacitance of an exemplary HRPS with different doping ratios of CNT;

FIG. 2D illustrates pressure response of relative capacitance change of an exemplary HRPS with different doping ratios of CNT;

FIG. 2E illustrates that approximately 0.5 wt % appears to be the optimal doping concentration and tangential sensitivities are labeled for this curve;

FIG. 2E illustrates a comparison of the sensitivity of an exemplary HRPS with capacitive pressure sensors reported in the literature in the pressure ranges of 1-5, 5-10, 10-30, and 30-50 kPa;

FIG. 2F illustrates cyclic response of an exemplary HRPS up to 5000 cycles from 0-5 kPa;

FIGS. 3A-3E, red and pink represent solid layers, greens represent nonconductive porous layers and blues represent conductive PNC; FIG. 3A is a photograph of four different dielectric materials with and without porosity and CNT-doping in Ecoflex™; FIG. 3B is a schematic illustration of the four conventional capacitive pressure sensors and HRPS; FIG. 3C illustrates pressure-strain curves of all five capacitive pressure sensors; FIG. 3D illustrates relative capacitance changes of all five capacitive pressure sensors; FIG. 3E illustrates normalized tangential sensitivity vs. pressure of all five capacitive pressure sensors where Arrow \textcircled{1} indicates enhancement due to porosity and Arrow @due to the hybrid response of conductive PNC;

FIG. 3F illustrates the sensitivities within the pressure ranges of various capacitive pressure sensors;

FIGS. 4A-4M are schematic illustrations of the electric fields of capacitive pressure sensors with (FIG. 4A) nonconductive PNC, (FIG. 4B) high-resistivity conductive PNC, and (FIG. 4C) low-resistivity conductive PNC; FIG. 4D illustrates simplified equivalent circuit of capacitive pressure sensor with nonconductive PNC; (FIG. 4E) theoretical capacitance and (FIG. 4F) relative capacitance changes for capacitive pressure sensor with nonconductive PNC of different dielectric constants; (FIG. 4G) simplified equivalent circuit of the HRPS with high-resistivity conductive PNC; (FIG. 4H) theoretical capacitance and (FIG. 4I) relative capacitance of the HRPS with conductive PNC of different resistance. FIGS. 4J-4M illustrate theoretical (solid curves) and experimental (dashed curves) capacitance change of the HRPS with the CNT doping ratio of (FIG. 4J) 0.25 wt %, (FIG. 4K) 0.5 wt %, (FIG. 4L) 0.75 wt %, and (FIG. 4M) 1 wt %; and

FIG. 5A illustrates detection of a 0.7-mg drosophila using a 1×1 cm² HRPS. The corresponding average effective pressure is 0.07 Pa; (FIG. 5B) Detection of air flow coming out of an air blower 3 cm above the sensor; (FIG. 5C) Detection of three successive waterdrops on the RPS; (FIG. 5D) Schematic illustration of carotid artery and temporal artery, and benchmarking waveforms of carotid arterial pulse (CAP) and temporal arterial pulse (TAP); (FIG. 5E) Photograph of the RPS installed on the skin over the carotid artery where a 3M Tegaderm™ tape was used for lamination, which induced a preload of 1.5 kPa; (FIG. 5F) Capacitance response of CAP from a breath-holding subject; (FIG. 5G Capacitance response of CAP from a subject with normal breaths; (FIG. 5H) Filtered CAP to eliminate respiratory signals from CAP; (FIG. 5I) Photographs of a subject with an HRPS on the frontal temporal artery wearing a virtual reality (VR) headset over the HRPS. The VR headset applied 8 kPa preload on the RPS; (FIG. 5J) Unfiltered capacitance response of RPS under the VR headset; (FIG. 5K) Filtered TAP showing characteristic TAP peaks.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers, or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

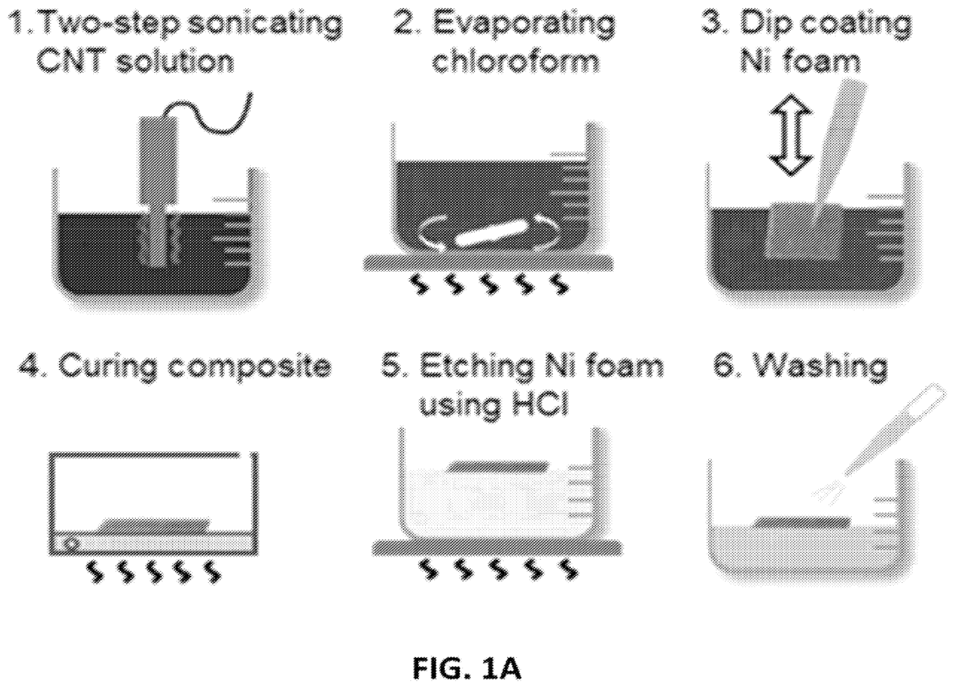

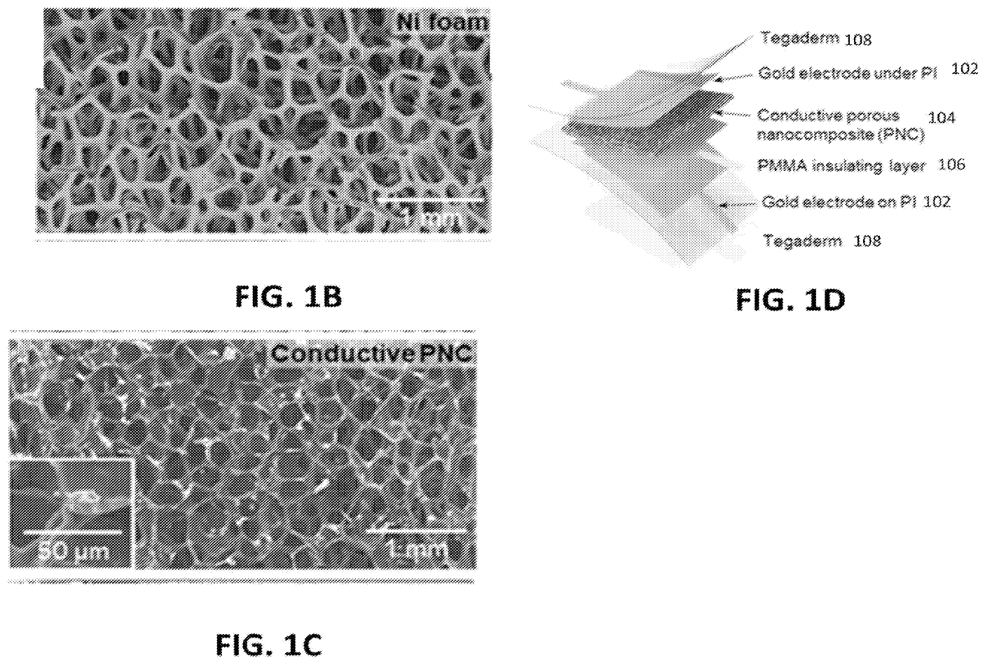

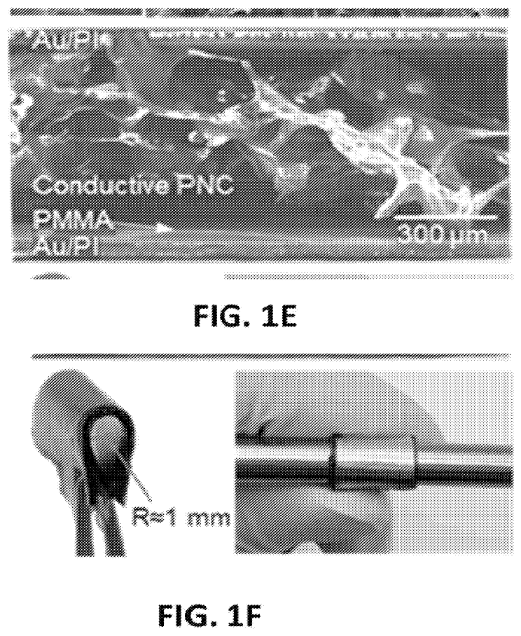

FIG. 1A illustrates an exemplary fabrication process for porous nano-composite (PNC) that can be used in the disclosed hybrid response capacitive sensor. As shown in FIG. 1A, at Step 1 a solution of hydroxyl-functionalized CNTs and chloroform is sonicated before and after adding an elastic rubber such as uncured Ecoflex™ for a uniform dispersion of the CNTs. T Step 2, the solution is heated and stirred to evaporate the chloroform until the weight ratio of chloroform and Ecoflex™ reaches 10 to 1. A 650-μm-thick nickel foam is used as the template for the PNC (see FIG. 1B) and at Step 3 is dipped into and then withdrawn from the solution mixture. At Step 4, the solution-coated nickel foam is heated at approximately 150° C. for 30 minutes to fully evaporate the chloroform and cure the CNT-doped Ecoflex™ nanocomposite. Afterwards, at Step 5, the sample is immersed in hydrochloric acid (HCl) to fully etch away the nickel. At Step 6, the leftover PNC is rinsed with distilled water. The final PNC has an approximately 86% porous open-cell structure with tubular ligaments as shown in FIG. 1C. The PNC is biocompatible due to the proven biocompatibility of Ecoflex™ and CNT nanocomposites.^{[68, 69]}

To construct an exemplary HRPS, a piece of PNC 104 is sandwiched by two flexible electrodes 102 with an ultrathin insulating layer 106 added between the PNC and one side of the electrodes 102 as illustrated in FIG. 1D. The electrode layers 102 are gold/polyimide (Au/PI) films and the insulating layer 106 is a polymethyl methacrylate (PMMA) film with a thickness of approximately 500 nm. In some instances, the multilayer assembly may be packaged between two transparent and flexible medical dressings (e.g., Tegaderm™, (3M, St. Paul, MN)) 108. Although the PNC 104 is electrically conductive, the PMMA film 106 acts as an insulating layer, thus making the whole pressure sensor capacitive. To prevent short circuits, the PMMA 106 is transferred onto the Au/PI 102 after the edges of the Au layer are slightly engraved by a laser cutter. A cross-sectional scanning electron microscope (SEM) image of the HRPS is shown in FIG. 1E. The air gaps surrounding the conductive ligaments produce parasitic capacitances. The packaged HRPS is flexible enough to be bent to a radius of 1 mm as shown in FIG. 1F. The estimated minimum bending radius is approximately 271 m.

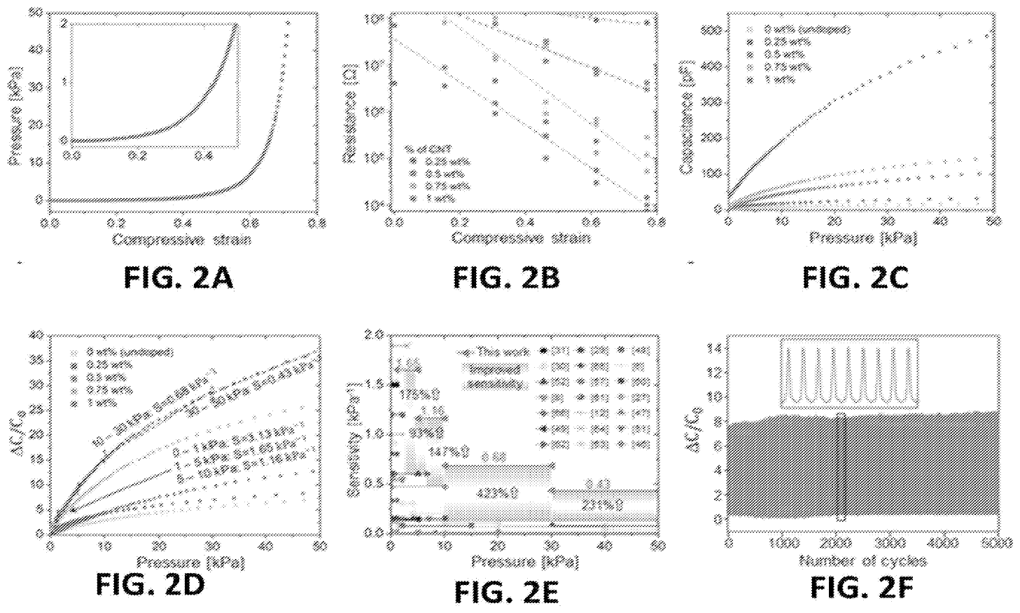

The flexibility of embodiments of the RPS can be best characterized by the nominal pressure-strain curve shown in FIG. 2A. The highly porous structure leads to a strong nonlinear response. The RPS is able to reach approximately 50% compressive strain under just 2 kPa pressure, as evident in the blown-up chart. The low initial compressive modulus is associated with the collapse of the PNC cells due to the elastic bending and buckling of the micro-ligaments.^{[70, 71]} The HRPS's high compliance is an important contributing factor to its high sensitivity. The electrical resistance vs. compressive strain curves are plotted in a semi-log chart in FIG. 2B. The weight concentration of CNT in the nanocomposite varies from approximately 0.25 wt % to approximately 1 wt %, and the resistance of the PNC is measured without the insulating layer. The resistance initially decreases by orders of magnitude upon compression due to the increasing contact between conductive ligaments in the PNC, and later, from the densification of the CNT network in the nanocomposite.^{[71]} As expected, the PNCs with greater CNT doping concentrations have lower initial and final resistance. The resistivities of the PNCs at 0.77 compressive strain are 53 MΩ·m, 2.5 MΩ·m, 100 kΩ·m, and 7.1 kΩ·m (approximately) when CNT doping ratios were 0.25, 0.5, 0.75, and 1 wt %, respectively. The piezoresistivity (slopes of the curves in FIG. 2B) varies depending on the CNT doping concentrations due to the nonlinear behaviors within the percolation zone.^{[72, 73]}

As used herein, “flexible” refers to properties of materials comprising the described sensors such that the sensors are capable of responding to an applied force by being flexed or bent without breaking; able to be turned, bowed, or twisted, without breaking; pliable; not stiff or brittle, while the sensors substantially maintain their original underlying shape while being flexed. This is contrasted to “stretchable,” where the underlying shape of the sensors changes in response to the applied force and may or may not return to the original shape after application of the force. For example, a sensor with a surface area of 15 cm will generally maintain the 15 cm surface area when being flexed, but the surface area of the sensor may increase when being stretched.

The capacitance changes of HRPSs with varying amounts of CNT in the PNC are plotted in FIG. 2C. In addition to the HRPS, a capacitive pressure sensor made out of undoped but porous Ecoflex™ was also included for comparison. Three samples were fabricated and tested for each CNT doping ratio, and the middle pressure response curve from the samples was selected to plot in FIG. 2C. The relative standard deviation among the three samples does not exceed 11% within the plotted pressure range. It is obvious that a greater CNT wt % results in a larger absolute capacitance change of the RPS upon compression. The monotonic dependence on CNT wt %, however, does not persist when the capacitance change is normalized by initial capacitance as shown in FIG. 2D. Due to an abrupt increase in the initial capacitance when the CNT doping is beyond 0.5 wt %, the RPS with 0.5-wt %-CNT PNC shows the highest sensitivity among the samples tested. Due to the existence of an optimal doping concentration, the HRPS is in distinct contrast to other capacitive pressure sensors, which always have higher sensitivities with larger amounts of conductive dopants which correspond to higher dielectric constants^{[56, 63, 64, 67, 74]}. The sensitivity of the HRPS with 0.5 wt % CNT changes with applied pressure and is found to be 3.13 kPa⁻¹ within 0-1 kPa, 1.65 kPa⁻¹ within 1-5 kPa, 1.16 kPa⁻¹ within 5-10 kPa, 0.68 kPa⁻¹ within 10-30 kPa, and 0.43 kPa⁻¹ within 30-50 kPa. The sensitivity of the HRPS exceeds that of other capacitive pressure sensors reported in the recent decade in the pressure range above 3 kPa (see FIG. 2E)^{[6, 8, 12, 27-31, 46-49, 51, 52, 57, 60-66]}. Notably, this improvement is most impressive in the large pressure regime, with a maximum of 423% within 10-30 kPa. Such enhancement is explained through detailed comparisons of HRPS with other types of capacitive pressure sensor and circuit models in the follows. The reversibility and durability of the HRPS are tested with 5000 repetitions of compression up to 5 kPa as shown in FIG. 2F. The relative capacitance change increases by 12% after 1000 cycles and increases by an additional 3% after 5000 cycles. Loading-unloading and cyclic tests are also carried out with pressures reaching 50 kPa, and the results show a slight increase of the baseline, which is due to irreversibly closed pores under repeated high compression.

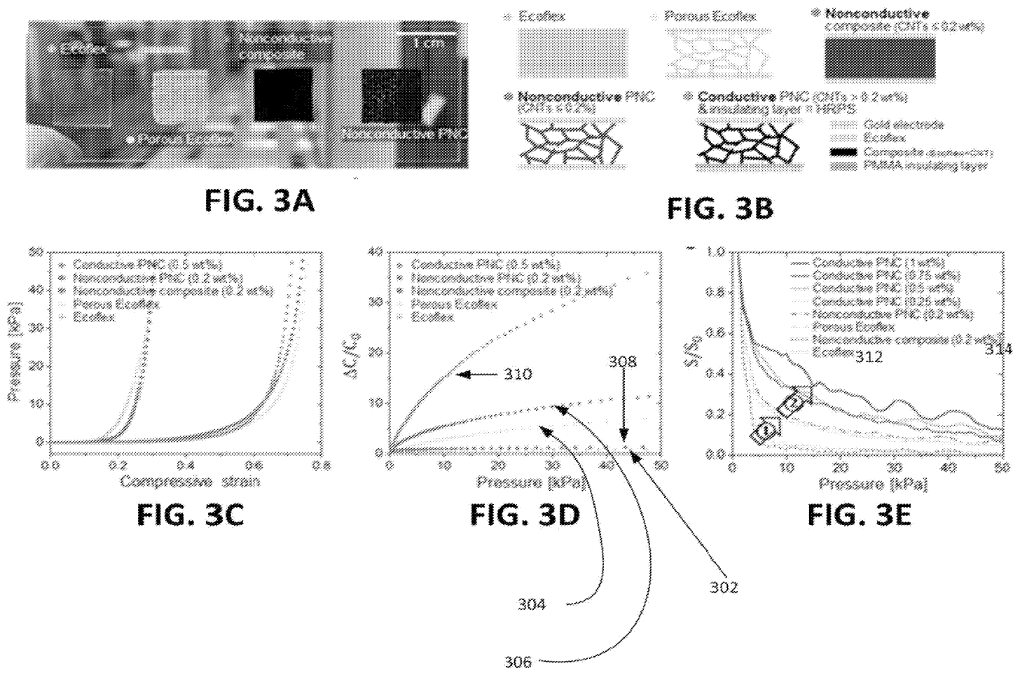

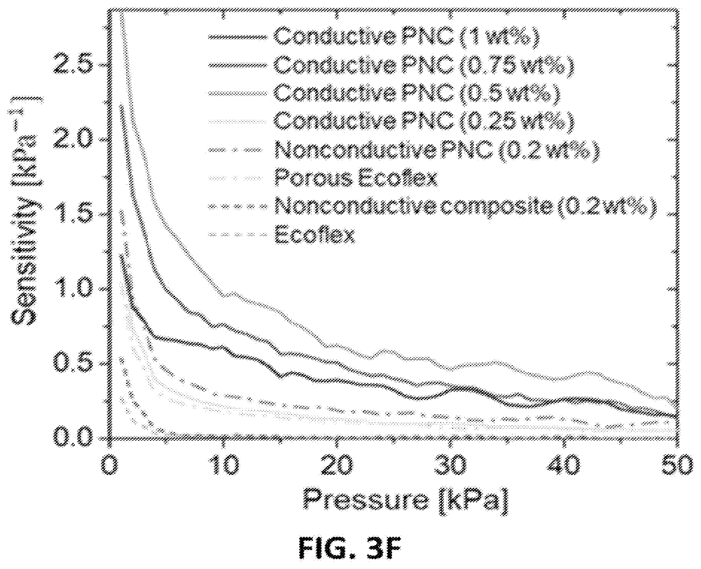

To compare performance of the disclosed HRPS, four capacitive pressure sensors made with conventional dielectric materials (undoped solid Ecoflex™, undoped porous Ecoflex™, doped but nonconductive solid composite, and doped nonconductive PNC) were fabricated as shown in FIG. 3A. All porous materials were fabricated using the same nickel foam template.

The 0.2 wt % CNT doping, which is just below the electrical percolation threshold, improves the dielectric constant of Ecoflex™ from 1.8 to 6.4. The four chosen dielectric layers well represent recent research advances in capacitive pressure sensors, which are adding air gaps (solid Ecoflex™ vs. porous Ecoflex™) and improving dielectric constant (solid Ecoflex™ vs. nonconductive solid composite, porous Ecoflex™ vs. nonconductive PNC). The performance of these four conventional capacitive pressure sensors is compared with the disclosed HRPS, which utilizes the conductive PNC with an insulating nanomembrane. 2D schematics of the conventional capacitive pressure sensors and HRPS are depicted in FIG. 3B.

FIG. 3C displays the pressure-strain curves of the five pressure sensors. Solids are clearly stiffer than porous materials, but the low CNT concentrations do not induce significant changes to the mechanical properties for either the solid or the porous materials.

The normalized capacitance vs. strain curves of the five different capacitive pressure sensors are shown in FIG. 3D, and the sensitivities within the pressure ranges of 0-1, 1-5, 5-10, 10-30, and 30-50 kPa are plotted in FIG. 3F. As expected, adding air gaps improves the sensitivity of the capacitive pressure sensor (pink (darker grey) 302 vs. pale green (lighter grey) 304 curves in FIG. 3D). Improving the dielectric constant of the ligaments in the porous dielectrics provide an additional significant enhancement to the sensitivity (pale green (lighter grey) 304 vs. green (darker grey) 306 curves in FIG. 3D). However, increasing the dielectric constant of the solid dielectrics is not as effective in elevating sensitivity (pink 302 vs. red 308 curves in FIG. 3D). In fact, the dielectric constant of a solid should not affect sensitivity if it remains constant during compression. In reality, however, the dielectric constant does increase slightly with compression, due to the densification of the CNT network.^{[67]} Among the four conventional capacitive pressure sensors, the one with nonconductive PNC (green curve) 306 demonstrates the largest sensitivity due to the synergistic effects of air gaps and high dielectric constant ligaments. This sensitivity can be considered an upper limit for capacitive pressure sensors using dielectric materials because the porosity (86%) and the CNT loading (0.2 wt %) are both close to their thresholds. The sensitivity of the HRPS (blue curve) 310 drastically surpasses all four conventional capacitive pressure sensors over all pressure ranges as evident in FIG. 3D.

To investigate the decay in sensitivity with increased pressure, the tangential sensitivities of every kPa of all five capacitive pressure sensors are measured against the applied pressure, and the normalized sensitivity (S/S_0) vs. pressure is displayed in FIG. 3E. Two major mechanisms for sensitivity enhancement, air gaps and the hybrid response, are indicated by the two arrows in the figure. First, when the dielectric material is solid (red 312 and pink 314 curves), with or without CNT doping, a sharp decrease of the normalized sensitivity occurs below 5 kPa. Therefore, these types of capacitive pressure sensors produce bilinear capacitance curves, which are also widely observed with other early published capacitive pressure sensors.^{[8, 31, 67]} The drastic drop of relative sensitivity is alleviated when the dielectric has a porous structure as demonstrated by Arrow \textcircled{1} in FIG. 3E. This improvement is achieved through the enhanced effective compliance of the porous dielectric material caused by the bending and buckling of the ligaments.^{[70]} Similar to the capacitive pressure sensor with solid dielectrics, the sensors with porous dielectrics exhibit the same tendency of relative sensitivity change with pressure independent of the CNT doping ratio, although their absolute sensitivities are very different. The second mechanism for the reduced declination of normalized sensitivity is attained through the hybrid response of the HRPS. For the RPS with PNC doped with 0.25 wt % CNT (barely conductive), the normalized sensitivity curve is very similar to those of the capacitive pressure sensor made out of porous Ecoflex™ or nonconductive PNC. This is due to the piezocapacitive response still dominating the impedance of this barely conductive PNC. As the amount of CNT increases and the PNC resistance lowers, the relative sensitivity trend is improved as demonstrated by Arrow\textcircled{2} in FIG. 3E. In conclusion, these two mechanisms, air gaps and hybrid responses, have distinctive effects in sustaining the sensitivity over a wide pressure range. However, increasing CNT doping can affect the absolute sensitivities but not necessarily the relative sensitivity.

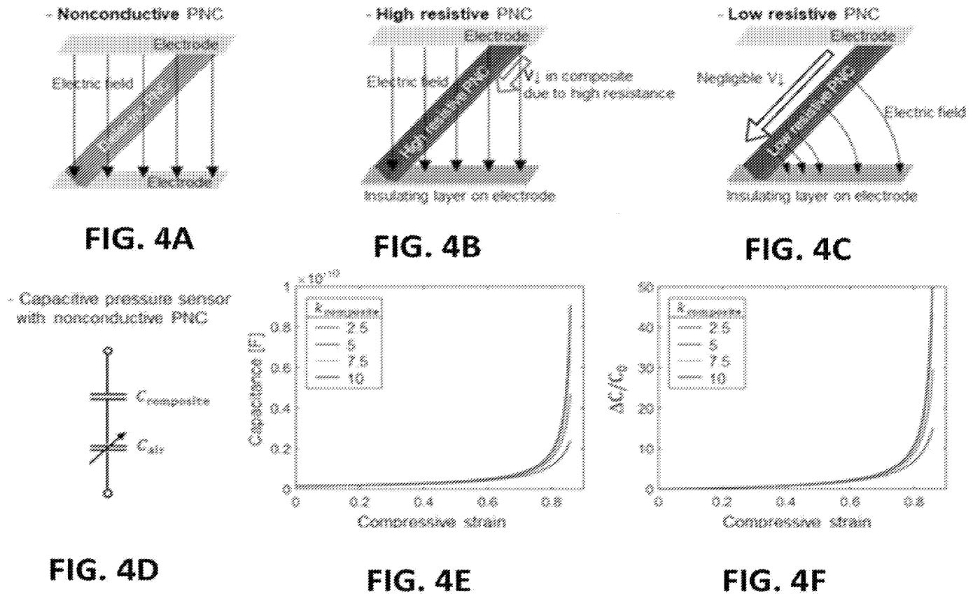

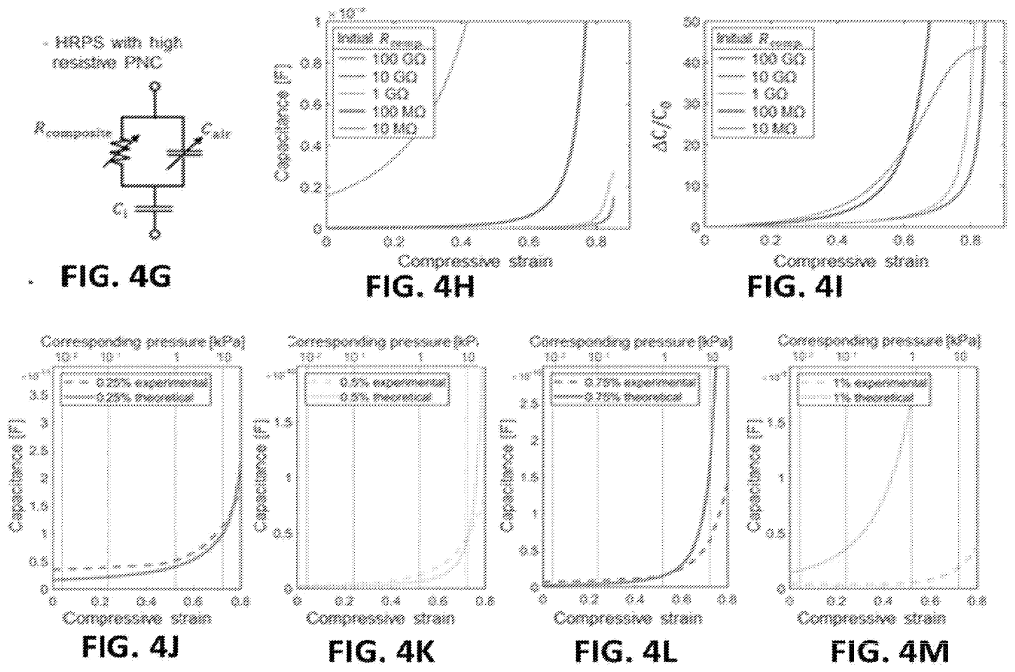

To offer a quantitative understanding of the sensing mechanism of the disclosed HRPS, the HRPS is modeled and compared to a conventional pressure sensor using simplified equivalent circuits. A single inclined ligament with the open space as air is used to represent the PNC in FIGS. 4A-4C. The equivalent circuit of a capacitive pressure sensor depends on the electrical property of the PNC. When the PNC ligaments are nonconductive and there is no added insulating layer, the electric field simply forms between the two parallel electrodes separated by a dielectric layer (see FIG. 4A). For the HRPS with a barely conductive PNC and an added insulating layer, there is a significant potential drop along the ligament, hence there is still a potential drop between the top electrode and the ligament (see FIG. 4B). For a HRPS with a more conductive PNC, the potential drop in the ligament becomes negligible such that there is no longer a potential drop between the top electrode and the ligament. In another word, the electric field only exists between the ligament and the bottom electrode, as illustrated in FIG. 4C. In this case, since some of the air gaps are no longer within (and thus affecting) the electric field, the pressure sensitivity is expected to be compromised.

Based on these electric field models, equivalent circuits of capacitive pressure sensors with nonconductive PNC and high-resistivity conductive PNC were built such that the global capacitance change could be analyzed. The equivalent circuit for the capacitive pressure sensor with nonconductive PNC is comprised of some capacitance from the nonconductive composite ligaments (C_{composite}) in series with some capacitance from air gaps (Cₐᵢᵣ). These capacitances are in series because the electric field passes through one after another as depicted in FIG. 4D. Cₐᵢᵣ and C_{composite} are defined in the following expressions:

$${C}_{air} = \frac{{k}_{air} \cdot {ε}_{0} \cdot A}{t ( {φ}_{0} - ϵ )} , \qquad ( 1 )$$

$${C}_{composite} = \frac{{k}_{composite} \cdot {ε}_{0} \cdot A}{t ( 1 - {φ}_{0} )} , \qquad ( 2 )$$

where k is the dielectric constant (kₐᵢᵣ=1), ε₀ is the permittivity of vacuum (∈₀=8.85×10⁻¹² F/m), A is the area of the capacitive pressure sensor (e.g. A=1×1 cm²), t is the initial thickness of the PNC (t=650 μm), φ₀ is the initial porosity of the nanocomposite (φ₀=0.86), and E is the nominal compressive strain. Assuming compression only affects the volume of air, only Cₐᵢᵣ is strain dependent. Using the equivalent circuit, global capacitance vs. compressive strain for various k_{composite} can be computed and is plotted in FIG. 4E. As k_{composite} increases from 2.5 (corresponding to k_{Ecoflex}) to 10 with increased CNT doping, the capacitance becomes more sensitive to strain, but the effect of k appears to saturate. The effect of k_{composite} on the relative capacitance change is similar as shown in FIG. 4F. This result verifies our previous observation that higher sensitivity is achieved with greater CNT doping in the nonconductive PNC.

The equivalent circuit for the HRPS with high-resistivity PNC is offered in FIG. 4G. The piezocapacitive response due to the reduction of air gap is modeled as Cₐᵢᵣ and the piezoresistive response due to the contacts and collapses of tubular ligaments as R_{composite}. Since the nanocomposite is conductive, we no longer have C_{composite}. Cₐᵢᵣ and R_{composite} are in parallel, and their combined impedance is in series to a fixed Cᵢ representing the insulating PMMA nanomembrane. The capacitance of air and insulating layer can be expressed as

$${C}_{air} = \frac{{k}_{air} \cdot {ε}_{0} \cdot A}{t ( {φ}_{0} - ε )} , \qquad ( 3 )$$

$${C}_{i} = \frac{{k}_{PMMA} \cdot {ε}_{0} \cdot A}{{t}_{PMMA}} , \qquad ( 4 )$$

where kₐᵢᵣ=1, k_{PMMA}=4, and t_{PMMA}=500 nm. The piezoresistive response of the conductive PNC can be fitted from our experimental measurements given in FIG. 4B:

$${R}_{composite} = \frac{{R}_{0}}{{φ}_{0}^{2}} {( ϵ - {φ}_{0} )}^{2} + \frac{{R}_{0}}{1000} , \qquad ( 5 )$$

where R₀ is the initial resistance of the conductive PNC before compression. Based on the circuit model, we calculate the capacitance vs. strain response of the HRPS with various R₀, which is plotted in FIG. 4H. The values of R₀ are hypothetical, but the range of R₀ is chosen to represent CNT concentrations from 0.25 wt % (R₀≈100 GΩ) to 1 wt % (R₀≈10 MΩ). As the initial resistance of the conductive PNC decreases, the initial capacitance of the RPS increases. Notably, the initial capacitance of the sensor soars when R₀ is lower than 100 MΩ. The jump of initial capacitance would significantly reduce the relative capacitance change, thus explaining the nonmonotonic effect of R₀ in FIG. 4I. Among the HRPSs with R₀ from 10 MΩ to 100 GΩ, the one with R₀=100 MΩ appears to have the highest sensitivity. This result agrees with our experimental finding that the optimal CNT loading with highest sensitivity is 0.5 wt % (see FIG. 2D). Comparing FIG. 4I with FIG. 4F, it is also obvious that the HRPS has a much higher sensitivity than the capacitive pressure sensor with nonconductive PNC.

Despite the fact that the theoretical results are obtained based on highly simplified equivalent circuit models, those results can be verified with experimental measurements. The analytical and experimental results for HRPSs with CNT doping ratios of 0.25 wt %, 0.5 wt %, 0.75 wt %, and 1 wt % are compared in FIGS. 4J-4M. The strong agreement between the two in FIGS. 4J and 4K indicates that the equivalent circuit represents the hybrid sensing mechanism of the HRPS well. The analytical and experimental results start to deviate when the CNT doping ratio is 0.75 wt % (see FIG. 4I) and eventually become unrelated when the CNT doping ratio reaches 1 wt % (see FIG. 4M). This could be explained by the schematic in FIG. 4C—if the high conductivity of the nanocomposite ligaments completely changes the electric field, the equivalent circuit in FIG. 4G is no longer applicable.

From both analytical calculation and experimental result, it is concluded that there is an optimum resistance of conductive PNC. It should be low enough to warrant a large overall capacitance change of the HRPS but not so small that the initial capacitance becomes outrageous. The normalized capacitance change of HRPS can be analytically expressed according to the circuit model of FIG. 4G. With the premise of Cᵢ>>Cₐᵢᵣ and taking a derivative with respect to R₀, the following analytical expression of the optimal R₀ is arrived at,

$${R}_{{0}_{optimum}} = 7.7 \; 839 {ω}^{- 1} {C}_{{air}_{0}}^{- \frac{3}{4}} {C}_{i}^{- \frac{1}{4}} , \qquad ( 6 )$$

which indicates that the optimal R₀ is dictated by Cₐᵢᵣ, Cᵢ, and the frequency at which the capacitance is measured (ω). Plugging in the experimental values of ω=2π·1000 rad/sec, Cₐᵢᵣ_{₀}=1.58 pF, and Cᵢ=7.08 nF, it is estimated that R₀_{ₒₚₜᵢₘᵤₘ}=96 MΩ, which agrees with our observation that R₀_{ₒₚₜᵢₘᵤₘ}=100 MΩ in FIG. 4I. Consequently, as long as the geometry, porosity, insulating material, and measurement frequency of the HRPS are specified, the optimal resistance of the PNC can be easily identified using Equation (6).

Although the theoretical analysis based on the highly simplified circuit model is able to offer a basic explanation for the hybrid effects and to predict the optimal CNT doping concentration, it still has room to improve. For example, the geometry of the pores was not considered in this model. Under the same porosity, the size and number of pores could vary greatly from sample to sample. In the case of very small pores and dense ligaments of the PNC, the R_{composite} and Cₐᵢᵣ cannot be modeled as simply connected in parallel. Our model also omitted electrical contact resistance (ECR). With irregular contact between the PNC and the Au electrodes, the ECR is significant and cannot be neglected.^{[77]}

### Examples/Results

PNC fabrication: A mixture of hydroxyl functionalized multi-wall carbon nanotubes (Carbon Nanotubes Plus) and chloroform (Sigma-Aldrich) was prepared with a ratio of 1 mg CNT:2 chloroform. For 0.25-wt %-CNT-doped PNC, the ratio was 1:3 considering the dilution ratio of Ecoflex™ in the chloroform. The solution was sonicated by a sonicator (Q500, QSonica) with a power of 500 watts for 10 minutes. Uncured Ecoflex™ (Ecoflex™ 00-30, Smooth-on, Inc.) base polymer was then added to the solution according to the target doping ratio of CNT, and the new mixture was sonicated for 10 minutes. After sonication, the solution was heated and stirred at 100° C. and 400 rpm using a magnetic hotplate stirrer (Fisher Scientific) to evaporate the chloroform. When the chloroform to Ecoflex™ weight ratio reached 10:1, a nickel foam (Tₘₐₓ) was dipped into the solution for 5 seconds and then extracted and put in a 150° C. oven for 30 minutes to fully evaporate the chloroform and cure the CNT-Ecoflex™ composite. The whole specimen was immersed in a 3M HCl (hydrochloric acid, Sigma-Aldrich) at 80° C. for 12 hours to etch the nickel foam template. Finally, the PNC was washed with distilled water.

Dielectric materials fabrication: Nonporous Ecoflex™: Ecoflex™ was molded in a 1 cm×1 cm×650 μm CNC-machined PTFE (Polytetrafluoroethylene, McMaster-Carr) mold and cured in the oven at 150° C. for 30 minutes. Porous Ecoflex™: a nickel foam was dipped into a 10:1 diluted Ecoflex™ by chloroform for 5 seconds. After that, the process followed that of PNC. Nonporous nanocomposite: the solution of CNT and chloroform was sonicated twice before and after adding Ecoflex™ for 10 minutes, and the chloroform was fully evaporated with stirring at 600 rpm. The leftover composite of CNT and Ecoflex™ was molded in the PTFE mold and cured in an oven at 150° C. for 30 minutes.

Fabrication of 500-nm-thick insulating layer: PVA (Polyvinyl Alcohol solution, Flinn Scientific) was spin-coated on the silicon wafer at 1000 rpm for 45 seconds and baked at 70° C. for 1 minute as a releasing agent. PMMA (PMMA A4, MicroChem) was spin-coated on the PVA at 300 rpm for 45 seconds and baked at 180° C. for 2 minutes. When immersed in a deionized water bath, the PVA layer was dissolved and the PMMA film floated to the surface of water. A temporary tattoo paper (Silhouette temporary tattoo paper, Silhouette) was used to pick up the PMMA film and the PMMA/tattoo paper was dried on a hot plate at 50° C. for 30 min. The PMMA could be transferred to other surfaces by smearing water to the tattoo paper.

HRPS fabrication: A 100-nm-gold-on-13-μm-polyimide (PI) bilayer (Sheldahl) was tailored into the desired electrode design. The gold layer was engraved by a width of 100 μm from all edges through laser beam machining (ProtoLaser U4, LPKF). The Au/PI electrode was attached to a 3M Tegaderm™ tape and the insulating PMMA layer was transferred onto the electrode. Finally, the conductive PNC was sandwiched between a Au/PI/Tegaderm film and a PMMA/Au/PI/Tegaderm film.

Compression testing: A Dynamic Mechanical Analyzer (RSA-G2, TA Instruments) was used to control and measure the applied pressure and displacement. The resistance of the conductive PNC was measured by a digital multi-meter (DM3068, Rigol), and the impedance of the HRPS was measured by an LCR meter (3532-50, Hioki) at 1 kHz frequency with a 2 V AC signal, both in situ.

Calculation of PNC porosity: After measurement of the weight and volume of the PNC, porosity was calculated based on the density of Ecoflex™ and CNT.

Definition of electrical percolation threshold: The traditional definition of electrical percolation threshold is the point at which the direct current (DC) conductivity rises the most rapidly with increases in filler concentrations. In our research, we measured the material's electrical impedance under alternating current (AC) to obtain both capacitance and resistance. We defined the electrical percolation threshold by the phase of the impedance. A nanocomposite containing resistive and reactive components has a phase angle between 0° (purely resistive) and −90° (purely capacitive). The porous composite was determined to be conductive if its phase angle was measured to be between 0° and −3° during compression up to 50 kPa.

Calculation of sensitivity of pressure sensors every kPa: After calculating the normalized capacitance of pressure sensors, they were 1-D interpolated by pressure to get enough data points for an accurate sensitivity determination. Then, the sensitivity in every kPa was calculated using linear regression and smoothing via moving average filter.

Measurement of arterial pulses: The arterial pulses were measured while the subject lied supine on a bed. For the detection of carotid arterial pulses, the subject rotated their neck by 45°.

Experiments on Human Subjects: All measurement on human subjects were conducted under approval from the Institutional Review Board of the University of Texas at Austin (protocol number: 2015-05-0024). Informed consent was obtained from all subjects involved in the study.

Several experiments were designed to demonstrate the high sensitivity of the RPS over a wide pressure range. Subtle pressures were applied on the HRPS in three different ways: without preload, with 1.5 kPa preload, and with 8 kPa preload.

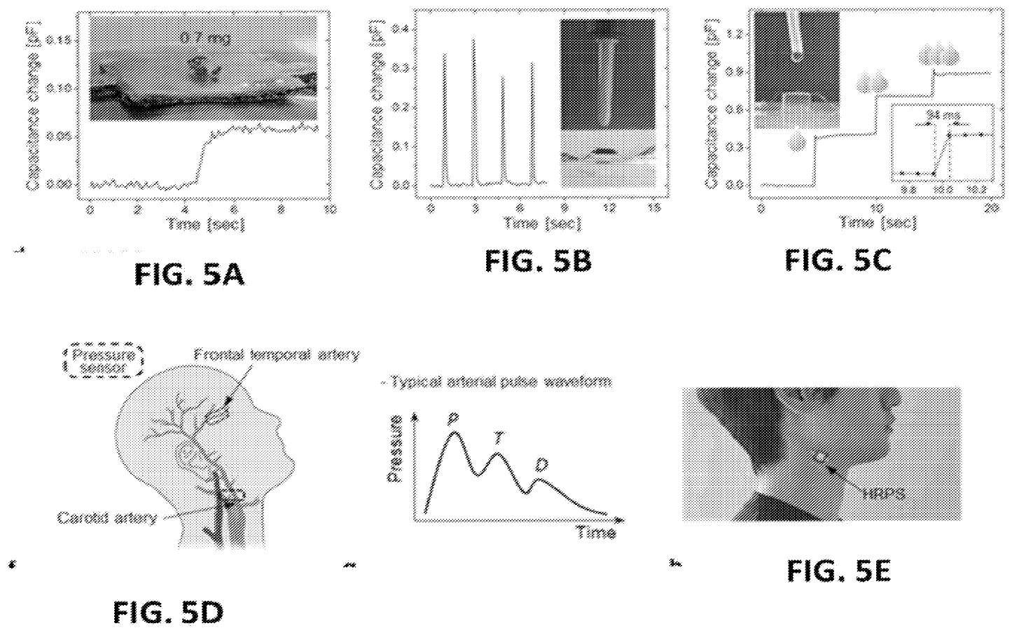

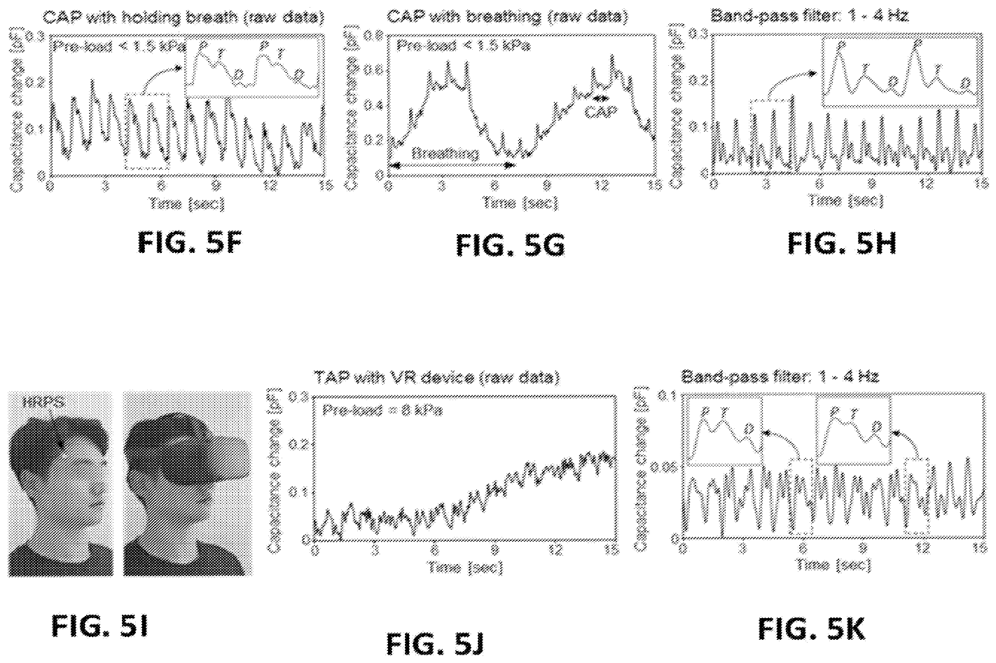

Firstly, tiny pressures that our skin experiences in daily life, such as the landing of a fly, a breeze, and water drops, were applied to the HRPS without preload. In FIG. 5A, a 1×1 cm² HRPS detected the pressure applied by a 0.7-mg drosophila, which corresponded to an effective average pressure of 0.07 Pa. In FIG. 5B, the RPS was able to sense breezes of air from an air blower 3 cm above the sensor. In FIG. 5C, the HRPS could differentiate the pressures induced by three water droplets applied one after another. It demonstrated a response time of 94 ms, which was the time resolution of our LCR meter (Hioki 3532-50).

Secondly, we laminated a HRPS on human skin with preload to measure pulse waveforms. The carotid artery and the frontal temporal artery have well-known subtle pulsations^{[79]} that require high sensitivity devices to detect. FIG. 5D exhibits the location of the carotid artery and the temporal artery with benchmark arterial pulse waveforms obtained by medical gold standards such as invasive arterial lines.^{[80-84]} For the carotid arterial pulse (CAP), a HRPS was placed on the neck over the carotid artery packaged between two medical tapes (3M Tegaderm™), which induced a preload of around 1.5 kPa over the HRPS as shown in FIG. 5E. While the human subject held his/her breath, the CAP was clearly visible without any amplification or signal processing (FIG. 5F). When the subject was breathing, respiration was visible as low-frequency undulations superimposed on the CAP as shown in FIG. 5G. After applying a band-pass filter from 1 to 4 Hz, the CAP signal in FIG. 5H appears similar to that in FIG. 5F.

To induce a larger preload to the HRPS, the subject put on a virtual reality (VR) headset to cover a HRPS applied at the frontal temporal artery as shown in FIG. 5I. The VR headset applied a preload of about 8 kPa over the HRPS. Even under a large preload, the unprocessed signal in FIG. 5J demonstrates that heart rate measurements can be obtained from the temporal arterial pulse (TAP). After filtering the data using the same band-pass filter (1-4 Hz), a typical TAP waveform can be clearly observed in FIG. 5K. To the best of our knowledge, this is the first capacitive pressure sensor able to detect the TAP. In addition to the CAP and TAP, the more widely measured radial arterial pulse (RAP) was also detectable by placing an exemplary packaged HRPS on the wrist.

Finally, to demonstrate the HRPS's capabilities for detecting high pressure, we attached the HRPS to a 80 kg subject's planta and measured the pressure from footsteps on a soft yoga mat. The maximum footstep pressure recorded through the HRPS was 125 kPa and is comparable with other reported footstep pressures.^{[85]}

### CONCLUSIONS

Although ultra-high sensitivity in flexible capacitive pressure sensors has been achieved before, declining sensitivity with increasing pressure is a well-known and long-standing challenge. Described herein is a flexible capacitive pressure sensor with high sensitivity over wide pressure ranges. By sandwiching an electrically conductive and highly porous nanocomposite and an ultrathin solid insulating layer between two parallel electrodes, the disclosed capacitive pressure sensor benefits from the combined piezoresistive and piezocapacitive responses of the PNC. The disclosed RPS achieved a sensitivity of 3.13 kPa⁻¹ within 0-1 kPa, 1.65 kPa⁻¹ within 1-5 kPa, 1.16 kPa⁻¹ within 5-10 kPa, 0.68 kPa⁻¹ within 10-30 kPa, and 0.43 kPa⁻¹ within 30-50 kPa pressure ranges. By comparing the HRPS with four conventional capacitive pressure sensor counterparts, we successfully differentiated the contribution of air gaps from the hybrid responses. We established and experimentally validated a theoretical model which successfully unveils the sensing mechanism of the HRPS and analytically determines the optimal PNC resistance controllable by tuning the CNT doping. We demonstrated the sensitivity of the HRPS by detecting subtle pressure changes with various preloads. Even when the RPS was covered by a VR headset, we were able to obtain the first noninvasive measurement of the TAP with a skin mounted pressure sensor. The wide sensing range was manifested by plantar pressure sensing. In addition to pulse waveform sensing, the exemplary flexible RPS is also promising for many other potential uses in prosthesis, tactile sensing, and e-skin for surgical or soft robots.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

### REFERENCES

Throughout this application, various publications are referenced. The disclosures of these publications in their entireties are hereby incorporated by reference into this application in order to more fully describe the state of the art to which the methods and systems pertain. These publications include
[1] S.-H. Sunwoo, K.-H. Ha, S. Lee, N. Lu, D.-H. Kim, Annu. Rev. Chem. Biomol. Eng. 2021, 12, 359.
[2] S. Xiang, D. Liu, C. Jiang, W. Zhou, D. Ling, W. Zheng, X. Sun, X. Li, Y. Mao, C. Shan, Adv. Funct. Mater. 2021, 2100940.
[3] Y. Tang, H. Zhou, X. Sun, N. Diao, J. Wang, B. Zhang, C. Qin, E. Liang, Y. Mao, Adv. Funct. Mater. 2020, 30, 1907893.
[4] C. M. Boutry, M. Negre, M. Jorda, O. Vardoulis, A. Chortos, O. Khatib, Z. Bao, Sci. Robot. 2018, 3, eaau6914.
[5] M. Liu, X. Pu, C. Jiang, T. Liu, X. Huang, L. Chen, C. Du, J. Sun, W. Hu, Z. L. Wang, Adv. Mater. 2017, 29, 1703700.
[6] Y. Wan, Z. Qiu, J. Huang, J. Yang, Q. Wang, P. Lu, J. Yang, J. Zhang, S. Huang, Z. Wu, C. F. Guo, Small 2018, 14, e1801657.
[7] H.-H. Chou, A. Nguyen, A. Chortos, J. W. To, C. Lu, J. Mei, T. Kurosawa, W.-G. Bae, J. B.-H. Tok, Z. Bao, Nat. Commun. 2015, 6, 1.
[8] S. C. Mannsfeld, B. C. Tee, R. M. Stoltenberg, C. V. H. Chen, S. Barman, B. V. Muir, A. N. Sokolov, C. Reese, Z. Bao, Nat. Mater. 2010, 9, 859.
[9] Y. Gao, H. Ota, E. W. Schaler, K. Chen, A. Zhao, W. Gao, H. M. Fahad, Y. Leng, A. Zheng, F. Xiong, Adv. Mater. 2017, 29, 1701985.
[10] K. Meng, J. Chen, X. Li, Y. Wu, W. Fan, Z. Zhou, Q. He, X. Wang, X. Fan, Y. Zhang, Adv. Funct. Mater. 2019, 29, 1806388.
[11] S. Lee, A. Reuveny, J. Reeder, S. Lee, H. Jin, Q. Liu, T. Yokota, T. Sekitani, T. Isoyama, Y. Abe, Nat. Nanotechnol. 2016, 11, 472.
[12] H. Kim, G. Kim, T. Kim, S. Lee, D. Kang, M. S. Hwang, Y. Chae, S. Kang, H. Lee, H. G. Park, Small 2018, 14.
[13] M. Kaltenbrunner, T. Sekitani, J. Reeder, T. Yokota, K. Kuribara, T. Tokuhara, M. Drack, R. Schwödiauer, I. Graz, S. Bauer-Gogonea, Nature 2013, 499, 458.
[14] C. L. Choong, M. B. Shim, B. S. Lee, S. Jeon, D. S. Ko, T. H. Kang, J. Bae, S. H. Lee, K. E. Byun, J. Im, Adv. Mater. 2014, 26, 3451.
[15] C. Pang, G.-Y. Lee, T.-i. Kim, S. M. Kim, H. N. Kim, S.-H. Ahn, K.-Y. Suh, Nat. Mater. 2012, 11, 795.
[16] J. H. Kim, J. Caprioli, J. Ophthalmic Vis. Res. 2018, 13, 170.
[17] C. Robba, D. Cardim, T. Tajsic, J. Pietersen, M. Bulman, J. Donnelly, A. Lavinio, A. Gupta, D. K. Menon, P. J. Hutchinson, PLoS Med. 2017, 14, e1002356.
[18] Y. Zang, F. Zhang, C.-a. Di, D. Zhu, Mater. Horiz. 2015, 2, 140.
[19] Z. Li, Z. L. Wang, Adv. Mater. 2011, 23, 84.
[20] C. Lou, S. Wang, T. Liang, C. Pang, L. Huang, M. Run, X. Liu, Materials 2017, 10, 1068.
[21] K. He, Y. Hou, C. Yi, N. Li, F. Sui, B. Yang, G. Gu, W. Li, Z. Wang, Y. Li, Nano Energy 2020, 104743.
[22] Z. Li, F. Qiang, S. Dai, S. Shen, Y. Huang, N.-J. Huang, G. Zhang, L.-Z. Guan, J. Gao, Y. Song, Nanoscale 2019.
[23] Y. Lee, J. Park, S. Cho, Y.-E. Shin, H. Lee, J. Kim, J. Myoung, S. Cho, S. Kang, C. Baig, ACS nano 2018, 12, 4045.
[24] M. T. Chorsi, E. J. Curry, H. T. Chorsi, R. Das, J. Baroody, P. K. Purohit, H. Ilies, T. D. Nguyen, Adv. Mater. 2019, 31, 1802084.
[25] J. Chun, K. Y. Lee, C. Y. Kang, M. W. Kim, S. W. Kim, J. M. Baik, Adv. Funct. Mater. 2014, 24, 2038.
[26] W. Choi, J. Lee, Y. Kyoung Yoo, S. Kang, J. Kim, J. Hoon Lee, Appl. Phys. Lett. 2014, 104, 123701.
[27] H. Shi, M. Al-Rubaiai, C. M. Holbrook, J. Miao, T. Pinto, C. Wang, X. Tan, Adv. Funct. Mater. 2019, 29, 1809116.
[28] S. R. A. Ruth, L. Beker, H. Tran, V. R. Feig, N. Matsuhisa, Z. Bao, Adv. Funct. Mater. 2019, 1903100.
[29] C. G. Nnnez, W. T. Navaraj, E. O. Polat, R. Dahiya, Adv. Funct. Mater. 2017, 27.
[30] L. Viry, A. Levi, M. Totaro, A. Mondini, V. Mattoli, B. Mazzolai, L. Beccai, Adv. Mater. 2014, 26, 2659.
[31] S. Park, H. Kim, M. Vosgueritchian, S. Cheon, H. Kim, J. H. Koo, T. R. Kim, S. Lee, G. Schwartz, H. Chang, Adv. Mater. 2014, 26, 7324.
[32] M. Ramuz, B. C. K. Tee, J. B. H. Tok, Z. Bao, Adv. Mater. 2012, 24, 3223.
[33] Y. Yamada, T. Morizono, Y. Umetani, H. Takahashi, IEEE Trans. Ind. Electron. 2005, 52, 960.
[34] V. Amoli, J. S. Kim, S. Y. Kim, J. Koo, Y. S. Chung, H. Choi, D. H. Kim, Adv. Funct. Mater. 2019, 1904532.
[35] Y. Chang, L. Wang, R. Li, Z. Zhang, Q. Wang, J. Yang, C. F. Guo, T. Pan, Adv. Mater. 2021, 33, 2003464.
[36] A. Chortos, J. Liu, Z. Bao, Nat. Mater. 2016, 15, 937.
[37] M. L. Hammock, A. Chortos, B. C. K. Tee, J. B. H. Tok, Z. Bao, Adv. Mater. 2013, 25, 5997.
[38] J. Li, R. Bao, J. Tao, Y. Peng, C. Pan, J. Mat. Chem. C 2018.
[39] H. Yousef, M. Boukallel, K. Althoefer, Sens. Actuators A 2011, 167, 171.
[40] N. Bai, L. Wang, Q. Wang, J. Deng, Y. Wang, P. Lu, J. Huang, G. Li, Y. Zhang, J. Yang, Nat. Commun. 2020, 11, 1.
[41] Q. Liu, Z. Liu, C. Li, K. Xie, P. Zhu, B. Shao, J. Zhang, J. Yang, J. Zhang, Q. Wang, Adv. Sci. 2020, 7, 2000348.
[42] S. H. Cho, S. W. Lee, S. Yu, H. Kim, S. Chang, D. Kang, I. Hwang, H. S. Kang, B. Jeong, E. H. Kim, S. M. Cho, K. L. Kim, H. Lee, W. Shim, C. Park, ACS Appl. Mater. Interfaces 2017, 9, 10128.
[43] B. Nie, R. Li, J. Cao, J. D. Brandt, T. Pan, Adv. Mater. 2015, 27, 6055.
[44] Z. Zhu, R. Li, T. Pan, Adv. Mater. 2018, 30, 1705122.
[45] S. Qiao, N. Lu, Int. J. Solids Struct. 2015, 58, 353.
[46] Y. Xiong, Y. Shen, L. Tian, Y. Hu, P. Zhu, R. Sun, C.-P. Wong, Nano Energy 2020, 70, 104436.
[47] J. Yang, S. Luo, X. Zhou, J. Li, J. Fu, W. Yang, D. Wei, ACS Appl. Mater. Interfaces 2019, 11, 14997.
[48] Y. Wan, Z. Qiu, Y. Hong, Y. Wang, J. Zhang, Q. Liu, Z. Wu, C. F. Guo, Adv. Electron. Mater. 2018, 4, 1700586.
[49] S. Baek, H. Jang, S. Y. Kim, H. Jeong, S. Han, Y. Jang, D. H. Kim, H. S. Lee, RSC Adv. 2017, 7, 39420.
[50] Q. Zhou, B. Ji, Y. Wei, B. Hu, Y. Gao, Q. Xu, J. Zhou, B. Zhou, J. Mater. Chem. A 2019, 7, 27334.
[51] J. C. Yang, J.-O. Kim, J. Oh, S. Y. Kwon, J. Y. Sim, D. W. Kim, H. B. Choi, S. Park, ACS Appl. Mater. Interfaces 2019, 11, 19472.
[52] D. Kwon, T.-I. Lee, M. Kim, S. Kim, T.-S. Kim, I. Park, presented at 2015 Transducers-2015 18th International Conference on Solid-State Sensors, Actuators and Microsystems (TRANSDUCERS), 2015.
[53] J. Hwang, Y. Kim, H. Yang, J. H. Oh, Compos. B Eng. 2021, 211, 108607.
[54] A. Chhetry, S. Sharma, H. Yoon, S. Ko, J. Y. Park, Adv. Funct. Mater. 2020, 30, 1910020.
[55] R. Y. Tay, H. Li, J. Lin, H. Wang, J. S. K. Lim, S. Chen, W. L. Leong, S. H. Tsang, E. H. T. Teo, Adv. Funct. Mater. 2020, 30, 1909604.
[56] J. Yang, D. Tang, J. Ao, T. Ghosh, T. V. Neumann, D. Zhang, E. Piskarev, T. Yu, V. K. Truong, K. Xie, Adv. Funct. Mater. 2020, 30, 2002611.
[57] W. Cheng, L. Yu, D. Kong, Z. Yu, H. Wang, Z. Ma, Y. Wang, J. Wang, L. Pan, Y. Shi, IEEE Electron Device Lett. 2018, 39, 1069.
[58] S. Lee, S. Franklin, F. A. Hassani, T. Yokota, M. O. G. Nayeem, Y. Wang, R. Leib, G. Cheng, D. W. Franklin, T. Someya, Science 2020, 370, 966.
[59] C. C. Vu, J. Kim, Sens. Actuators A 2020, 314, 112029.
[60] J. Kim, E. F. Chou, J. Le, S. Wong, M. Chu, M. Khine, Adv. Healthc. Mater. 2019, 1900109.
[61] Z. He, W. Chen, B. Liang, C. Liu, L. Yang, D. Lu, Z. Mo, H. Zhu, Z. Tang, X. Gui, ACS Appl. Mater. Interfaces 2018, 10, 12816.
[62] Y. Joo, J. Yoon, J. Ha, T. Kim, S. Lee, B. Lee, C. Pang, Y. Hong, Adv. Electron. Mater. 2017, 3, 1600455.
[63] S.-Y. Liu, J.-G. Lu, H.-P. D. Shieh, IEEE Sens. J. 2018, 18, 1870.
[64] F. Liu, F. Han, L. Ling, J. Li, S. Zhao, T. Zhao, X. Liang, D. Zhu, G. Zhang, R. Sun, Chem. Eur. J 2018, 24, 16823.
[65] S. Wan, H. Bi, Y. Zhou, X. Xie, S. Su, K. Yin, L. Sun, Carbon 2017, 114, 209.
[66] X. Guo, Y. Huang, X. Cai, C. Liu, P. Liu, Meas. Sci. Technol. 2016, 27, 045105.
[67] J. Wang, J. Jiu, M. Nogi, T. Sugahara, S. Nagao, H. Koga, P. He, K. Suganuma, Nanoscale 2015, 7, 2926.
[68] J. H. Kim, J.-Y. Hwang, H. R. Hwang, H. S. Kim, J. H. Lee, J.-W. Seo, U. S. Shin, S.-H. Lee, Sci. Rep. 2018, 8, 1.
[69] W. J. Bae, J. B. Choi, K. S. Kim, S. J. Kim, H. J. Cho, U. S. Ha, S. H. Hong, J. Y. Lee, S. W. Kim, Transl. Androl. Urol. 2015, 4.
[70] D. De Vries, Eindhoven University of Technology 2009.
[71] L. Gong, S. Kyriakides, W.-Y. Jang, Int. J. Solids Struct. 2005, 42, 1355.
[72] T. Zhai, D. Li, G. Fei, H. Xia, Compos. Part A Appl. Sci. Manuf. 2015, 72, 108.
[73] M. Panahi-Sarmad, M. Noroozi, M. Abrisham, S. Eghbalinia, F. Teimoury, A. R. Bahramian, P. Dehghan, M. Sadri, V. Goodarzi, ACS Appl. Electron. Mater. 2020, 2, 2318.
[74] M. Pruvost, W. J. Smit, C. Monteux, P. Poulin, A. Colin, npj Flex. Electron. 2019, 3, 1.
[75] Y. Si, L. Wang, X. Wang, N. Tang, J. Yu, B. Ding, Adv. Mater. 2017, 29, 1700339.
[76] R. Widdle Jr, A. Bajaj, P. Davies, Int. J. Eng. Sci. 2008, 46, 31.
[77] N. Khalili, H. Naguib, R. Kwon, Soft matter 2016, 12, 4180.
[78] L. Jin, A. Chortos, F. Lian, E. Pop, C. Linder, Z. Bao, W. Cai, Proc. Natl. Acad. Sci. 2018, 115, 1986.
[79] R. E. Curran, Arch. Ophthalmol. 1986, 104, 1756.
[80] G. Chen, C. Au, J. Chen, Trends Biotechnol. 2021.
[81] B. Campbell, Heart Lung 1997, 26, 204.
[82] H. K. Walker, W. D. Hall, J. W. Hurst, Clinical methods: the history, physical, and laboratory examinations, Butterworth-heinemann, 1990.
[83] A. Cournand, H. A. Ranges, Proc. Soc. Exp. Biol. Med. 1941, 46, 462.
[84] M.-M. Laurila, M. Peltokangas, K. L. Montero, T. Siponkoski, J. Juuti, S. Tuukkanen, N. Oksala, A. Vehkaoja, M. Mäntysalo, IEEE Sens. J. 2019, 19, 11072.
[85] J. Tao, M. Dong, L. Li, C. Wang, J. Li, Y. Liu, R. Bao, C. Pan, Microsyst. Nanoeng. 2020, 6, 1.
[86] Z. Ma, A. Wei, J. Ma, L. Shao, H. Jiang, D. Dong, Z. Ji, Q. Wang, S. Kang, Nanoscale 2018, 10, 7116.
[87] L. Yang, Y. Liu, C. D. Filipe, D. Ljubic, Y. Luo, H. Zhu, J. Yan, S. Zhu, ACS Appl. Mater. Interfaces 2019, 11, 4318.
[88] C. M. Tringides, N. Vachicouras, I. de Lázaro, H. Wang, A. Trouillet, B. R. Seo, A. Elosegui-Artola, F. Fallegger, Y. Shin, C. Casiraghi, Nat. Nanotechnol. 2021, 1.

### Scope

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A flexible hybrid response capacitive pressure sensor comprising:
an electrically conductive porous nanocomposite (PNC) layer having a first surface and a second surface, wherein the first surface and the second surface are separated by a thickness of the electrically conductive porous nanocomposite layer;
an insulating layer, wherein the insulating layer covers and is in contact with at least a portion of the first surface of the PNC layer;
a first electrode, wherein the first electrode is in contact with a top surface of the insulating layer; and
a second electrode, wherein the second electrode is in contact with at least a portion of the second surface of the PNC layer,
wherein the PNC layer exhibits hybrid piezoresistive and piezocapacitive responses, resulting in significantly enhanced sensitivities over wide pressure ranges.

2. The pressure sensor of claim 1, wherein the PNC layer is comprised of doped carbon nanotubes (CNT) and a low viscosity and flexible, strong, and elastic rubber, and ligaments of the PNC layer are electrically conductive due to adequate CNT doping.

3. The pressure sensor of claim 1, wherein the PNC layer is approximately 86% porous with an open cell structure that enables distributed parasitic capacitance.

4. The pressure sensor of claim 1, wherein first electrode and/or the second electrode are comprised of gold/polyimide (Au/P) films.

5. The pressure sensor of claim 1, wherein the insulating layer is comprised of a polymethyl methacrylate (PMMA) film.

6. The pressure sensor of claim 5, wherein the PMMA film has a thickness of approximately 500 nm.

7. The pressure sensor of claim 1, wherein the pressure sensor is packaged between two transparent and flexible medical dressings.

8. The pressure sensor of claim 1, wherein the thickness of the PNC layer is approximately 650 m.

9. The pressure sensor of claim 1, wherein the significantly enhanced sensitivities are more than 400%, and the wide pressure ranges are from 3.13 kPa-1 within 0-1 kPa to 0.43 kPa-1 within 30-50 kPa.

10. A method of fabricating a flexible hybrid response capacitive pressure sensor comprising:
forming an electrically conductive porous nanocomposite (PNC) layer having a first surface and a second surface, wherein the first surface and the second surface are separated by a thickness of the electrically conductive porous nanocomposite layer;
attaching an insulating layer to the first surface of the PNC layer, wherein the insulating layer covers and is in contact with at least a portion of the first surface of the PNC layer;
attaching a first electrode to a top surface of the insulating layer, wherein the first electrode is in contact with at least a portion of the top surface of the insulating layer; and
attaching a second electrode to the second surface of the PNC layer, wherein the second electrode is in contact with at least a portion of the second surface of the PNC layer,
wherein the PNC layer exhibits hybrid piezoresistive and piezocapacitive responses, resulting in significantly enhanced sensitivities over wide pressure ranges.

11. The method of claim 10, wherein the PNC layer is comprised of doped carbon nanotubes (CNT) and a low viscosity and flexible, strong, and elastic rubber, and ligaments of the PNC layer are electrically conductive due to adequate CNT doping.

12. The method of claim 11, wherein the PNC layer is formed by:
a solution of hydroxyl-functionalized CNTs and chloroform is sonicated before and after adding uncured flexible, strong, and elastic rubber for a uniform dispersion of the CNTs;
the solution is heated and stirred to evaporate the chloroform until a weight ratio of chloroform and the flexible, strong, and elastic rubber reaches 10 to 1;
a 650-μm-thick nickel foam substrate is used as a template for the PNC and is dipped into and then withdrawn from the solution mixture;
the solution-coated nickel foam substrate is heated at approximately 150° C. for 30 minutes to fully evaporate the chloroform and cure the CNT-doped flexible, strong, and elastic rubber nanocomposite;
the substrate is immersed in hydrochloric acid (HCl) to fully etch away the nickel and the leftover PNC is rinsed with distilled water,
wherein the PNC has an approximately 86% porous open-cell structure with tubular ligaments.

13. The method of claim 10, wherein the PNC layer has an open cell structure that enables distributed parasitic capacitance.

14. The method of claim 10, wherein first electrode and/or the second electrode are comprised of gold/polyimide (Au/PI) films.

15. The method of claim 10, wherein the insulating layer is comprised of a polymethyl methacrylate (PMMA) film.

16. The method of claim 15, wherein the PMMA film has a thickness of approximately 500 nm.

17. The method of claim 10, further comprising packaging the pressure sensor between two transparent and flexible medical dressings.

18. The method of claim 10, wherein the significantly enhanced sensitivities are more than 400%, and the wide pressure ranges are from 3.13 kPa-1 within 0-1 kPa to 0.43 kPa-1 within 30-50 kPa.
